(19) United States
(12) Patent Application Publication
(10) Pub. No.: US 2025/0079007 A1
(43) Pub. Date: Mar. 6, 2025

(54) System and Method for Performing a Multi-Step, Multi-Factorial Decision Analysis

(71) Applicants: Zachary Oatley, North Canton, OH (US); Jeremy Glasgow, Tempe, AZ (US)

(72) Inventors: Zachary Oatley, North Canton, OH (US); Jeremy Glasgow, Tempe, AZ (US)

(21) Appl. No.: 18/821,779

(22) Filed: Aug. 30, 2024

Related U.S. Application Data
(60) Provisional application No. 63/579,787, filed on Aug. 30, 2023.

(51) Int. Cl. G16H 50/20; G16H 10/20; G16H 10/60; G16H 15/00; G16H 50/30

(52) U.S. Cl. CPC G16H 50/20; G16H 10/20; G16H 10/60; G16H 15/00; G16H 50/30

(57) ABSTRACT

A method and system for detecting sleep apnea in patients using an electronic healthcare record (EHR) system. An API configured to communicate with the EHR system is installed and relevant data inputs for predicting sleep apnea determined. An initial risk assessment for sleep apnea is automatically performed for a patient based on their health history is performed when the patient is seen by a healthcare provider. A visual prompt is displayed to the healthcare provider if the patient is at an elevated risk for sleep apnea. The visual prompt may include a listing of additional tests needed, a numerical risk level, or allow the healthcare provider to schedule sleep apnea related appointments. The method may also involve calculating a patient risk level, providing feedback to improve the accuracy of the risk assessment, determining co-factors, and incorporating cost-benefit analysis in the risk assessment.

### CROSS REFERENCE TO RELATED APPLICATION

This application claims priority to provisional application Ser. No. 63/579,787, filed Aug. 30, 2023, which is incorporated herein by reference in its entirety.

### BACKGROUND OF THE INVENTION

### Field of the Invention

The present disclosure pertains generally to methods and systems for detecting risk in using existing records. More specifically, it relates to methods and systems for detecting sleep apnea in patients using existing health data from electronic healthcare record systems.

### Description of Related Art

Sleep apnea is a common sleep disorder characterized by repeated complete or partial interruptions in breathing throughout the sleep cycle leading to apnea (breathing cessation) or hypopnea (shallow breathing). These interruptions, which can occur up to hundreds of times a night, can lead to oxygen desaturation and disrupted sleep and a variety of health problems such as heart disease, stroke, and diabetes.

Sleep apnea is a significant public health issue due to its high prevalence and substantial economic burden. It affects approximately 12% of the U.S. adult population, with an estimated 80% of cases undiagnosed. The economic burden of undiagnosed sleep apnea in the United States is estimated to be $149.6 billion. This burden manifests in increased healthcare utilization, decreased workplace productivity, and a higher incidence of workplace and motor vehicle accidents. The disorder is linked to increased all-cause mortality and decreased quality of life due to its association with oxidative stress, systemic inflammation, and other physiological disturbances. Despite its prevalence and serious health consequences, sleep apnea often goes undiagnosed due to the lack of awareness among healthcare providers and the general public, as well as the inconvenience and cost of traditional diagnostic methods.

Currently, the detection of sleep apnea largely relies on patient-reported symptoms and overnight sleep studies, which can be expensive, time-consuming, and uncomfortable for patients. Health care providers frequently use a questionnaire known as the STOP-BANG questionnaire that asks about snoring, tiredness, observed apnea, pressure (elevated blood pressure), body mass index (BMI>35 kg/m²), age (>50 years old), neck circumference (>40 cm), and gender (are you male). Each yes answer scores one point. The total score helps to determine the risk of sleep apnea with a score of 0-2 indicating a low risk, a score of 3-4 indicating an intermediate risk, and a score of 5-8 indicating a high risk.

Healthcare providers do not use the STOP-BANG questionnaire in the course of standard care thus requiring patients to raise a sleep related concern or otherwise determine that a risk exists for the disorder. Patients often do not raise sleep related concerns such as tiredness or snoring because they are subjective and often normalized. To identify patients that do not raise sleep related complaints, physicians must devote time and attention away from other patient needs to initiate and manually assess the scoring. As a result, many patients with sleep apnea remain undiagnosed and untreated, leading to poor quality of life, loss of workplace productivity, motor vehicle accidents, and increased healthcare costs.

Although STOP BANG does provide insight into a patient's sleep apnea risk, its design, scoring, and cutoff points are arbitrary. The tool does not account for differences in the relative amount of risk contributed by each factor or confounding interactions between each factor. It also uses fixed cutoff points for numerical variables such as BMI. For example, a patient with a BMI of 34 kg/m² would not receive a point while a patient with a BMI of 35 and 50 would both receive one point. The point-based output is also subjective by nature where each point increase in score is not proportional to increased risk.

In addition, the current methods for managing patient health data in healthcare settings are often fragmented and inefficient. The data relevant to predicting sleep apnea, such as patient's health history, are typically scattered across different electronic healthcare record systems, making it difficult for healthcare providers to access and utilize the data effectively. This lack of integration and interoperability among healthcare record systems poses significant challenges to the early detection and treatment of sleep apnea.

Moreover, the current methods for assessing the risk of sleep apnea are often subjective and inconsistent, leading to variability in diagnosis and treatment. There is a need for a more objective and standardized method for assessing the risk of sleep apnea based on patient's health data. Such a method would improve the accuracy and consistency of sleep apnea diagnosis and facilitate early detection and treatment of the disorder, saving healthcare costs and improving patient health outcomes.

To reduce the complexity and length of the Detailed Specification, and to fully establish the state of the art in certain areas of technology, Applicants herein expressly incorporate(s) by reference all of the following materials identified in each numbered paragraph below. The incorporated materials are not necessarily “prior art.”
US20230238142A1
US20130172687A1
US20080146893A1
US20200178844A1
US20170332917A1
U.S. Pat. No. 7,162,294B2
U.S. Ser. No. 10/327,711B2
US20210282707A1
US20150164410A1
US20220125377A1

Applicant(s) believe(s) that the material incorporated above is “non-essential” in accordance with 37 CFR 1.57, because it is referred to for purposes of indicating the background of the invention or illustrating the state of the art. However, if the Examiner believes that any of the above-incorporated material constitutes “essential material” within the meaning of 37 CFR 1.57(c)(1)-(3), applicant(s) will amend the specification to expressly recite the essential material that is incorporated by reference as allowed by the applicable rules.

### BRIEF SUMMARY OF THE INVENTION

The present invention provides, among other things, a method for detecting sleep apnea in patients using existing health data from an electronic healthcare record system.

It is an object of the invention to provide a method of detecting an elevated risk of sleep apnea from commonly available data points without requiring health care providers to independently identify an elevated risk.

It is an object of the invention to provide a software plugin that automatically notifies healthcare providers of an elevated risk of sleep apnea based on a patient's electronic health record, or otherwise provide a non-intrusive visual prompt for further sleep apnea evaluation alongside other patient data.

It is another object of the invention to provide a software program that uses both a patient's electronic health record and other data stored in a healthcare provider's customer relationship management software to identify an elevated risk of sleep apnea.

It is another object of the invention to provide a system that allows healthcare providers to more easily assess a patient for sleep apnea.

It is another object of the invention to provide prompts to healthcare providers to more quickly and easily determine if a patient is at an elevated risk for sleep apnea.

The above and other objectives may be achieved using methods involving determining the type of electronic healthcare record system from a variety of such systems, and installing an API configured to communicate with the determined system type. Data inputs in the healthcare record system that are relevant to predicting sleep apnea in a patient are identified and assessed. When a patient is being seen by a healthcare provider, an initial risk assessment for sleep apnea is automatically performed based on the patient's health history. A visual prompt is then displayed to the healthcare provider, informing them that the patient is at an elevated risk for sleep apnea. In some embodiments, the method further includes calculating a patient risk level for the patient and displaying the visual prompt to the healthcare provider when the patient risk level is greater than a selected value. The risk may appear alongside patient health summary data, other preventive care needs such as vaccinations, or alongside other risk assessments.

The visual prompt displayed to the healthcare provider may be a popup that is consistent with the electronic healthcare record type. The popup may prompt the healthcare provider to enter follow-up information that is relevant to determining whether the patient has sleep apnea, and any responses to the prompts are entered into the healthcare record system. In some implementations, the visual prompt comprises a listing of additional tests that could be used alone or in combination to determine whether the patient has sleep apnea or includes a numerical risk level for the patient. The visual prompt may also allow the healthcare provider to schedule sleep apnea related appointments for the patient.

Feedback may be provided to improve the accuracy of the initial risk assessment. The feedback may be provided in a machine learning algorithm that allows the system to constantly improve its ability to accurately determine risk. The system continually assesses available variables in the EHR or other data that is predictive of sleep apnea. Variables that do not strongly predict sleep apnea may be removed reducing the complexity and computing power needed for the model. Various machine learning models (like support vector machines, neural networks, and linear regression) are tested. Each model undergoes a recursive feature elimination process, which systematically removes less important features to find the best combination of predictors. The best features are then used to optimize the model's hyperparameters, ensuring the model performs as well as possible. The model may be retrained or adapted to predict different outcomes, such as using a different cutoff for sleep apnea severity or applying a different method for measuring the apnea-hypopnea index (AHI).

In certain embodiments, the initial risk assessment uses comorbid conditions such as COPD or high blood pressure to determine which patients are most likely to experience adverse events related to their sleep apnea or the differential benefit of providing patients with additional testing such as polysomnography or home sleep apnea testing. The initial risk assessment may be displayed as the total mortality risk attributable to sleep apnea. In some embodiments, the notice would provide information about the clinical benefit provided by sleep testing denominated in years of life lost, quality adjusted life years, or increase in mortality. This analysis could also be quantified economically by assigning economic values to the machine-learning predicted clinical outcomes and directly compared with the total cost of testing and treatment versus a monetary value that is assigned to the determined clinical benefit. The total cost and total benefit for the combined probabilities of various intermediate events may be adjusted to account for a combined expected value for a given action such as ordering a diagnostic sleep test.

Cost benefit analysis may be conducted using a longitudinal dataset that tracks patients' sleep apnea severity and along with a wide range of basic health outcomes. This dataset would be used to train a machine learning algorithm that predicts health outcomes for patients with untreated sleep apnea. By comparing the likelihood of these outcomes with those of patients without sleep apnea and patients who have received treatment, the extent to which diseases attributable to sleep apnea can be prevented is determined.

This extent of prevention may be represented as a ratio between zero and one where one means no prevention (i.e., the intervention has no effect), and zero means that the intervention completely prevents all sleep apnea related diseases. The total economic benefit is then calculated by multiplying the burden of sleep apnea by: the degree to which these diseases are preventable, the economic cost associated with the burden, the risk of having sleep apnea, and rate of treatment compliance.

As data is entered in the second part of the risk prediction, the visual prompt updates in real time to display new information regarding the patient's risk and the cost-benefit tradeoff. When the described condition is met, the visual prompt is altered in a manner that directs the healthcare provider to initiate further action such as diagnostic testing. This approach allows the model to assess whether the screening and treatment protocol is beneficial based on the likelihood of various outcomes rather than assuming that every screened patient will receive and benefit from treatment.

The method may further include offering a software program that performs the acts of the method, and offering the software program for sale on an EHR marketplace. The healthcare provider may be incentivized to perform the initial risk assessment based on revenue gained from increased testing volumes for sleep apnea.

Aspects and applications of the invention presented here are described below in the drawings and detailed description of the invention. Unless specifically noted, it is intended that the words and phrases in the specification and the claims be given their plain, ordinary, and accustomed meaning to those of ordinary skill in the applicable arts. The inventors are fully aware that they can be their own lexicographers if desired. The inventors expressly elect, as their own lexicographers, to use only the plain and ordinary meaning of terms in the specification and claims unless they clearly state otherwise and then further, expressly set forth the “special” definition of that term and explain how it differs from the plain and ordinary meaning. Absent such clear statements of intent to apply a “special” definition, it is the inventors' intent and desire that the simple, plain and ordinary meaning to the terms be applied to the interpretation of the specification and claims.

The inventors are also aware of the normal precepts of English grammar. Thus, if a noun, term, or phrase is intended to be further characterized, specified, or narrowed in some way, then such noun, term, or phrase will expressly include additional adjectives, descriptive terms, or other modifiers in accordance with the normal precepts of English grammar. Absent the use of such adjectives, descriptive terms, or modifiers, it is the intent that such nouns, terms, or phrases be given their plain, and ordinary English meaning to those skilled in the applicable arts as set forth above.

Further, the inventors are fully informed of the standards and application of the special provisions of 35 U.S.C. § 112 (f). Thus, the use of the words “function,” “means” or “step” in the Detailed Description or Description of the Drawings or claims is not intended to somehow indicate a desire to invoke the special provisions of 35 U.S.C. § 112 (f), to define the invention. To the contrary, if the provisions of 35 U.S.C. § 112 (f) are sought to be invoked to define the inventions, the claims will specifically and expressly state the exact phrases “means for” or “step for, and will also recite the word “function” (i.e., will state “means for performing the function of [insert function]”), without also reciting in such phrases any structure, material or act in support of the function. Thus, even when the claims recite a “means for performing the function of . . . ” or “step for performing the function of . . . ,” if the claims also recite any structure, material or acts in support of that means or step, or that perform the recited function, then it is the clear intention of the inventors not to invoke the provisions of 35 U.S.C. § 112 (f). Moreover, even if the provisions of 35 U.S.C. § 112 (f) are invoked to define the claimed inventions, it is intended that the inventions not be limited only to the specific structure, material or acts that are described in the preferred embodiments, but in addition, include any and all structures, materials or acts that perform the claimed function as described in alternative embodiments or forms of the invention, or that are well known present or later-developed, equivalent structures, material or acts for performing the claimed function.

### BRIEF DESCRIPTION OF THE SEVERAL VIEWS OF THE DRAWINGS

A more complete understanding of the present invention may be derived by referring to the detailed description when considered in connection with the following illustrative figures. In the figures, like reference numbers refer to like elements or acts throughout the figures.

FIG. 1 depicts a graphical flowchart of the method provided in one embodiment of the invention.

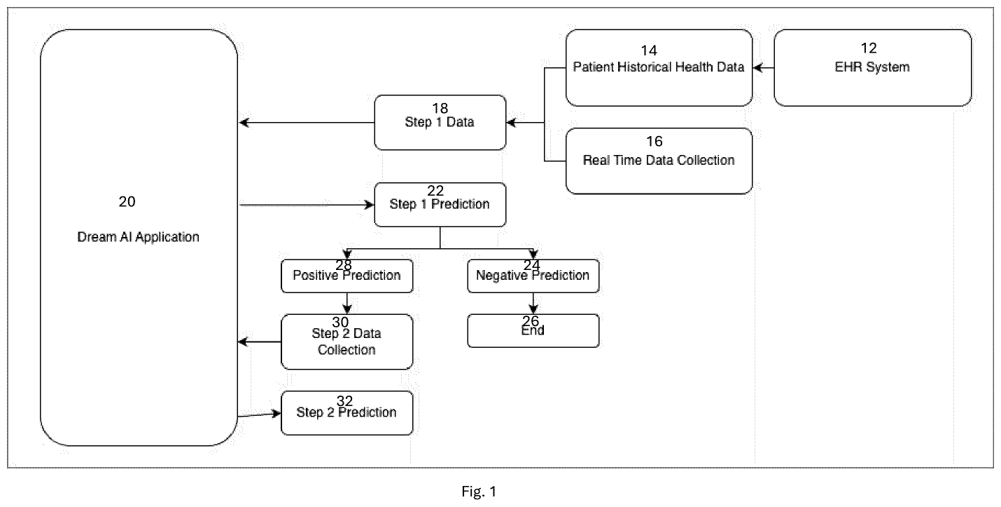

FIG. 2 depicts a graphical flowchart of a feedback loop for improving the predictive accuracy of a model in one embodiment of the invention.

Elements and acts in the figures are illustrated for simplicity and have not necessarily been rendered according to any particular sequence or embodiment.

### DETAILED DESCRIPTION OF THE INVENTION

In the following description, and for the purposes of explanation, numerous specific details are set forth to provide a thorough understanding of the various aspects of the invention. It will be understood, however, by those skilled in the relevant arts, that the present invention may be practiced without these specific details. In other instances, known structures and devices are shown or discussed more generally to avoid obscuring the invention. In many cases, a description of the operation is sufficient to enable one to implement the various forms of the invention, particularly when the operation is to be implemented in software. It should be noted that there are many different and alternative configurations, devices, and technologies to which the disclosed inventions may be applied. The full scope of the inventions is not limited to the examples that are described below.

In one application of the invention, a method for detecting sleep apnea for a healthcare provider from existing patient health data on an electronic healthcare record (EHR) system is provided. The EHR system 12 is a digital version of a patient's paper chart and includes comprehensive data about the patient's health history, diagnoses, treatments, medications, immunizations, allergies, radiology images and laboratory results. Many types of EHRs are common in the marketplace including EHR systems from Epic Systems, Cerner, Allscripts, Meditech, Athenahealth, NextGen Healthcare, eClinicalWorks, GE Healthcare (Centricity), Kareo, DrChrono, and the like. The different EHR systems may have different inputs, data tags, and graphical user interfaces. The choice of an EHR system often depends on the specific needs of the healthcare provider, including the size of the practice, specialty requirements, and budget.

Healthcare providers may also have a customer relationship management (CRM) system. The CRM system is typically a software system used to manage interactions with current and potential patients. The CRM system may include demographic and other data related to a patient to improve business relationships, streamline processes, and enhance service or marketing to patients.

An application programming interface (API) is a set of rules and protocols that allows different software applications to communicate with each other. APIs define the methods and data formats that applications use to interact, allowing them to request and exchange information or services. A type of EHR system is first determined and an API selected to allow the system to interact with the healthcare provider's EHR system 12. The data inputs 18 that are included in the EHR system are identified as relevant to predicting sleep apnea in a patient. Data inputs may include patient historical health data 14 including the patient's gender, height, weight, blood pressure history, past medical conditions, family medical history, medication history, vital signs, lab results and other historical patient characteristics, complaints, and diagnoses. The data inputs 18 may also include real-time data collection 16 collected by the EHR as a normal function of the patient visit.

When a patient is being seen by the healthcare provider, the data inputs 18 are collected by in an application 20 to provide an initial risk assessment 22 for sleep apnea for the patient. The initial risk assessment 22 is automatically performed based on the health history 14 for the patient that is included in the EHR and/or CRM. The initial risk assessment 22 is a predictive model that uses the identified data inputs to calculate the patient's risk of having sleep apnea. The risk assessment could be based on a variety of predictive models, including but not limited to, logistic regression, decision trees, random forests, and neural networks. The risk assessment may be a binary determination that the patient is or is not at an elevated risk for sleep apnea, a numerical risk score such as a score or percentage that indicates the patient's risk of having sleep apnea, or a separation of the patient into a pool of very low risk, low risk, intermediate risk, elevated risk, high risk, or very high risk.

If the prediction of the risk assessment 22 is a negative prediction 24, the process is ended and no visual prompt is provided to the healthcare provider. If the risk assessment results in a positive prediction 28, a visual prompt is displayed to the healthcare provider informing the healthcare provider that the patient is at an elevated risk for sleep apnea while the patient is being seen by the healthcare provider. The visual prompt may be a popup that is consistent with the electronic healthcare record type, matching the look and feel of the EHR system. The popup may prompt the healthcare provider to enter follow-up information 30 that is relevant to determining whether the patient has sleep apnea. For example, the popup may prompt the healthcare provider to ask any of the questions of the STOP-BANG questionnaire that are not already present in the EHR data. In a particular embodiment, the popup may be a dashboard for risk assessment that guides the healthcare provider through additional steps to determine the patient's potential diagnosis. Any responses to the prompts are entered into the healthcare record system and used to update the determination of risk of sleep apnea of the patient. The risk for sleep apnea is then determined again 32 based on the additional data collection 30.

The data collection 16 of the system may include providing an API configured to communicate with CRM software used by the healthcare provider that has additional information about the plurality of patients. Any CRM data or other additional information that is relevant to predicting sleep apnea in the patient is identified, and the identified relevant additional information is used when performing the initial risk assessment 22 or the final risk assessment 32. The additional information could include, but is not limited to, the patient's demographic, lifestyle factors, and any other relevant information. The initial risk assessment 22 may be used to determine whether to interact with the CRM to request any additional information 30. If the initial risk assessment indicates that the patient may be at an elevated risk of having sleep apnea, the additional information may be identified and used in the risk assessment. If the initial risk assessment indicates that the patient is at a low risk of having sleep apnea, the additional information may not be identified or used in the risk assessment.

An example of the model used in connection with the invention is provided below. The model can be adapted based on the datasets available, practice setting, and in other ways that would be apparent to an individual of reasonable skill in the art. Various methods for measuring sleep apnea incidence exist, each utilizing different cutoffs and severity assessment approaches. The model may be optimized to any specified target. The described methods are adaptable and can be modified to accommodate more current or practice-specific datasets using established data analytics techniques.

The algorithm training process uses patient data to predict sleep apnea risk. A two-factor analysis is conducted to exclude poorly predictive variables, reducing computational complexity before more rigorous feature selection methods are applied. This process uses the mean information coefficient, with a cutoff value selected to include all potentially relevant features based on a review of the literature and domain expertise. Further exclusion of variables is based on practical considerations such as availability in electronic medical records (EMR) and ease of data collection.

Rigorous feature selection methods are then applied to various machine learning models, including support vector machines, neural networks, multiple linear regression, and others. Recursive feature elimination is utilized to optimize the feature set while simultaneously optimizing the models' hyperparameters. Once this phase is completed, the machine learning model underpinning the second part of the prediction process, which incorporates sleep-specific data, is finalized.

Then, an additional model may be trained by eliminating sleep specific features from this feature set to only include features that are included routinely within in the EHR, and do not include sleep-specific data. This model is to be used in the initial prediction while the model above is used in the second risk prediction. Sleep specific features might include questions about snoring, a neck size measurement, or other useful data that is included in the training dataset, but not routinely collected in the user's health record. Then, using forward selection, additional features that are not sleep-specific and were first eliminated in the previous selection process are selected while simultaneously optimizing the models' hyperparameters. This process reoptimizes the selected features for this predictor once sleep-specific features are eliminated.

Once the selected feature sets for these two predictors are optimized for a specific target based on the AHI severity cutoff and AHI measurement method, additional models can be trained and optimized to additional targets. Information about the patient's risk for each cutoff or method may be included in the visual prompt as is deemed useful, according to provider preferences, professional recommendations, and insurance coverage determinations.

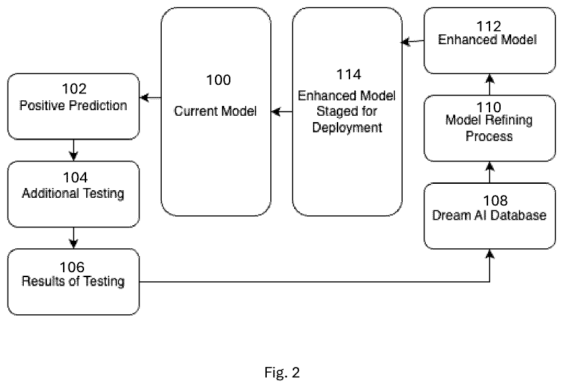

As shown in FIG. 2, the method may further comprise providing feedback to improve the accuracy of the initial risk assessment. The current model 100 provides an initial assessment. Upon a positive prediction 102 additional testing may be employed 104 to confirm or disprove the initial positive prediction. The results of that testing 106 goes into the system database 108 and the model is refined at 110. The enhanced model 112 is staged for deployment as an updated current model 114. The feedback could be based on the healthcare provider's observations and assessments, the patient's feedback, and any other relevant information. The feedback is used to update and refine the predictive model used in the initial risk assessment. In this process, data from tested patients is incorporated into the model training set to refine the predictions such that they remain accurate on diverse and evolving patient populations.

The initial risk assessment may determine co-factors that would make sleep apnea more harmful for a patient with the co-factors and use a cost-benefit analysis to determine whether to display the visual prompt to the healthcare provider. The co-factors could include, but are not limited to, the patient's age, weight, smoking history, alcohol consumption, and any other relevant health history.

In a particular embodiment, the cost-benefit analysis begins by dividing the dataset into two groups: a treatment group of patients who received sleep apnea treatment and a control group of patients who did not receive treatment. Two models are developed: one to predict each patient's risk of sleep apnea and another to estimate their personalized mortality risk using Cox regression analysis. The mortality risk model includes variables such as age, diabetes status, sex, smoking status, total cholesterol, HDL, systolic blood pressure, and the Apnea-Hypopnea Index (AHI), aligning with established cardiovascular risk scoring methods.

After training the mortality risk model, it is applied to the treatment group to estimate the relative hazard of cardiovascular mortality over time for each patient under two scenarios: with and without treatment. This allows for individualized recommendations rather than estimating the years of life lost (YLL) saved for the entire group. The difference in hazard ratio between the treated and untreated scenarios is calculated for each patient, providing a personalized estimate of the potential benefits of treatment.

The cost-effectiveness analysis may then be reframed to consider the net expected value for each patient, accounting for the probability of various intermediate events. Specifically, not every patient who is screened will receive treatment; this depends on their individualized sleep apnea risk. The analysis starts with the cost of screening (which may be, for example, $150 per patient) and considers the likelihood that a patient identified as high-risk will proceed to treatment. The cost of treatment is based on the price of a CPAP device.

The cost-effectiveness analysis may then be reframed to consider the net expected value for each patient, accounting for the probability of various intermediate events. Specifically, not every patient who is screened will receive treatment; this depends on their individualized sleep apnea risk. The analysis starts with the cost of screening (which may be, for example, $150 per patient) and considers the likelihood that a patient identified as high-risk will proceed to treatment. The cost of treatment is based on the price of a CPAP device.

To represent the net expected costs and benefits mathematically, the following variables are defined:
C_screen=Cost of screening ($150)
P_treatment=Predicted sleep apnea risk (based on the model's output)×Probability that a high-risk patient will initiate treatment after screening.
C_treatment=Cost of treatment (CPAP) ($500 to $1,000)
ΔHazard=Reduction in cardiovascular mortality hazard due to treatment
V_YLL=Value of a year of life saved ($150,000)
YLL_saved=Years of life saved per patient based on the change in estimated hazard
B_treatment=Expected benefit of treatment in monetary terms
The net expected costs are calculated as:
Net Expected Costs=C_screen+P_treatment×C_treatment
The net expected benefits are calculated as:
Net Expected Benefits=P_treatment×(ΔHazard×YLL_saved×V_YLL)

The net expected costs are calculated as:
Net Expected Costs=C_screen+P_treatment×C_treatment

The net expected benefits are calculated as:
Net Expected Benefits=P_treatment×(ΔRisk×YLLsaved×V_YLL)

Finally, the decision rule to determine if the screening and treatment protocol is cost-effective for an individual patient is:
Net Expected Benefits≥Net Expected Costs

If the net expected benefits exceed or equal the net expected costs, then the screening and treatment protocol is considered cost-effective for that patient.

The cost-benefit analysis could consider the potential costs of determining whether the patient has sleep apnea relative to the potential benefit to the patient of accurately determining whether the patient has sleep apnea. The notice may comprise a listing of additional tests needed to determine whether the patient has sleep apnea. The additional tests could include, but are not limited to, a sleep study, a physical examination, a medical history review, and any other relevant tests.

The method may further comprise offering a software program that performs the acts of the method. The software program could be offered for sale on an EHR marketplace. The software program could be a standalone program, or it could be integrated into the healthcare provider's existing system. The method may further comprise incentivizing the healthcare provider to perform the initial risk assessment based on revenue gained from increased testing volumes for sleep apnea. The incentive could be a financial incentive, a performance-based incentive, or any other type of incentive. The potential benefit to the patient may be at least partially determined based on additional predicted patient life years.

The additional predicted patient life years could be quality adjusted life years. Quality adjusted life years are a measure of the state of health of a person or group in which the benefits, in terms of length of life, are adjusted to reflect the quality of life. One QALY is equal to 1 year of life in perfect health. The initial risk assessment may be made in real time based on new data collected by the healthcare provider. The new data could include, but is not limited to, the patient's current symptoms, the results of any recent tests, and any other relevant information.

The visual prompt may allow the healthcare provider to schedule sleep apnea related appointments for the patient. The appointments could include, but are not limited to, a sleep study, a follow-up consultation, a treatment planning session, and any other relevant appointments.

In closing, it is to be understood that although aspects of the present specification are highlighted by referring to specific embodiments, one skilled in the art will readily appreciate that these disclosed embodiments are only illustrative of the principles of the subject matter disclosed herein. Therefore, it should be understood that the disclosed subject matter is in no way limited to a particular methodology, protocol, and/or reagent, etc., described herein. As such, various modifications or changes to or alternative configurations of the disclosed subject matter can be made in accordance with the teachings herein without departing from the spirit of the present specification. Lastly, the terminology used herein is for the purpose of describing particular embodiments only and is not intended to limit the scope of the present disclosure, which is defined solely by the claims. Accordingly, embodiments of the present disclosure are not limited to those precisely as shown and described.

Certain embodiments are described herein, including the best mode known to the inventors for carrying out the methods and devices described herein. Of course, variations on these described embodiments will become apparent to those of ordinary skill in the art upon reading the foregoing description. Specifically, the risk assessment may be used in connection with other health diagnoses or disorders or other areas where an initial determination of risk may be determined from existing systems data. Accordingly, this disclosure includes all modifications and equivalents of the subject matter recited in the claims appended hereto as permitted by applicable law. The terms “including” and “such as” are not limiting and should be interpreted as “including, but not limited to,” and “such as, for example,” respectively. Moreover, any combination of the above-described embodiments in all possible variations thereof is encompassed by the disclosure unless otherwise indicated herein or otherwise clearly contradicted by context.

We claim:

1. A method for detecting sleep apnea for a healthcare provider from existing patient health data on an electronic healthcare record system having a health history for each of a plurality of patients, the system comprising the acts of:
determining the electronic healthcare record system type from a plurality of electronic healthcare record system types;
installing an API configured to communicate with the electronic healthcare record type determined;
identifying the data inputs in the healthcare record system type that are relevant to predicting sleep apnea in a patient;
when a patient of the plurality of patients is being seen by the healthcare provider, automatically performing an initial risk assessment for sleep apnea for the patient based on the health history for the patient;
displaying a visual prompt to the healthcare provider informing the healthcare provider that the patient is at an elevated risk for sleep apnea while the patient is being seen by the healthcare provider.

2. The method of claim 1 further comprising the acts of:
providing an API configured to communicate with patient management software used by the healthcare provider that has additional information about the plurality of patients;
identifying any data in the additional information that is relevant to predicting sleep apnea in the patient;
using any identified relevant additional information when performing the risk assessment.

3. The method of claim 1 wherein the visual prompt is a popup that is consistent with the electronic healthcare record type and user-preferences and may appear beside patient summary data.

4. The method of claim 3 wherein the visual prompt provides risk information or cost benefit information that prompts the healthcare provider to collect and enter follow-up information that is relevant to determining whether the patient has sleep apnea and enters any responses to the prompts into the healthcare record system.

5. The method of claim 1 further comprising the acts of calculating a patient risk level for the patient and displaying or changing the appearance of the information in the visual prompt to the healthcare provider when the patient risk level is greater than a selected value.

6. The method of claim 2, wherein the initial risk assessment is performed and used to determine whether to identify the additional information.

7. The method of claim 1 further comprising providing feedback to improve the accuracy of the initial risk assessment.

8. The method of claim 1 wherein patient health history is used to determine the benefit of an intervention such as diagnostic testing in the patient to perform a cost-benefit analysis on whether the intervention is cost effective where the cost is the overall expected value of costs associated with the intervention including diagnosis and treatment and the benefit is the overall economic benefit of performing the intervention.

9. The method of claim 8 where the overall cost-benefit ratio is used to augment the information prompt according to user-specified or prespecified cutoff ratio.

10. The method of claim 8 wherein the cost-benefit analysis incorporates a determination of potential costs of determining whether the patient has sleep apnea relative to the potential savings in health care if the patient is diagnosed with sleep apnea versus health care costs if the patient has sleep apnea but is undiagnosed.

11. The method of claim 1 wherein the visual prompt comprises a listing of additional tests needed to determine whether the patient has sleep apnea.

12. The method of claim 1 wherein the visual prompt includes a numerical risk level for the patient.

13. The method of claim 1 further comprising offering a software program that installs the API when installed by the health care provider.

14. The method of claim 13 further comprising offering the software program for sale on an EHR marketplace.

15. The method of claim 1 further comprising incentivizing the healthcare provider to perform the initial risk assessment based on revenue gained from increased testing volumes for sleep apnea.

16. The method of claim 8 wherein the potential benefit to the patient is at least partially determined based on additional predicted patient life years.

17. The method of claim 16 wherein the additional predicted patient life years are quality adjusted life years.

18. The method of claim 1 wherein the initial risk assessment is made in real time based on new data collected by the healthcare provider.

19. The method of claim 1 wherein the visual prompt allows the healthcare provider to schedule sleep apnea related appointments for the patient or order diagnostic tests.

20. The method of claim one where the predictions may be calculated locally instead of communicating them through an API.
